(19) United States
(12) Patent Application Publication
(10) Pub. No.: US 2017/0232155 A1
(43) Pub. Date: Aug. 17, 2017

(54) THERMO-MECHANICAL PROCESSING OF HIGH ENTROPY ALLOYS FOR BIOMEDICAL APPLICATIONS

(71) Applicant: University of North Texas, Denton, TX (US)

(72) Inventor: Aleksandra Fortier, Coppell, TX (US)

(21) Appl. No.: 15/433,142

(22) Filed: Feb. 15, 2017

Related U.S. Application Data
(60) Provisional application No. 62/295,860, filed on Feb. 16, 2016.

(51) Int. Cl. A61L 31/02; C21D 9/00; C21D 1/18; C22C 30/00

(52) U.S. Cl. CPC A61L 31/022; C22C 30/00; C21D 9/0068; C21D 1/18

(57) ABSTRACT

The present invention includes an apparatus and method of making the apparatus that biomedical apparatus comprises a device adapted for use in a biomedical application that comprises a high entropy alloy comprising at least 5 elements.

### CROSS-REFERENCE TO RELATED APPLICATIONS

This application claims priority based on U.S. Provisional Application No. 62/295,860, filed Feb. 16, 2016. The contents of which is incorporated by reference in its entirety.

### TECHNICAL FIELD OF THE INVENTION

The present invention relates in general to the field of biomedical applications of novel materials, and more particularly, to novel biomechanical devices using high entropy alloys.

### STATEMENT OF FEDERALLY FUNDED RESEARCH

None.

### BACKGROUND OF THE INVENTION

Without limiting the scope of the invention, its background is described in connection with cardiovascular disease.

Heart disease is the leading cause of death among Americans with about 600,000 deaths per year and costs the United States about $110 billion each year.

Cardiovascular disease is often related to atherosclerosis, which can cause narrowing, rupture or erosion of the arterial wall, with eventual reduction or blockage of blood flow in the artery. Stent implant procedure is the insertion of a metal and/or plastic tubular structure that has the ability to expand into a cylindrical shape, either by use of balloon or a self-expansion mechanism.

### SUMMARY OF THE INVENTION

The present invention includes devices and methods of making devices that include high entropy alloys. Specifically, the present invention includes medical devices that comprise high entropy alloys that include at least five (5) elements. These elements can be included in near-equal molar ratios, e.g., CoCrFeNiMn, but can also include five (5) elements that are not equimolar, e.g., Al_{0.1}CoCrFeNi. In certain aspects, the materials are treated after being cast, e.g., by being rolled and heat-treated. These materials show increased strength and better micro-hardness distribution along weld zones.

In one embodiment, the present invention includes a biomedical apparatus comprising a device adapted for use in a biomedical application that comprises a high entropy alloy comprising at least 5 elements. In one aspect, the device is a stent, a valve, a plate, a plaque, a screw, a spring, a tube, a filter, an insert, one or more wires, or a joint. In another aspect, the high entropy alloy is selected from at least one of CoₓCrₓFeₓNiₓMnₓ, or AlₓCoₓCrₓFeₓNiₓ, wherein x is 0.05 to 2. In another aspect, the high entropy alloy is at least one or cold rolled, sintered, heat-treated, or microhardened. In another aspect, the high entropy alloy is heat-treated, rolled, and then heat-treated, wherein the first and second heat-treatment are for the same time and same temperature, or for a different time and temperature. In another aspect, the high entropy alloys are selected from Al_{0.1}CoCrFeNi or CoCrFeNiMn. In another aspect, the five (5) elements are about equimolar. In another aspect, the device is a composite of more than one high entropy alloy. In another aspect, the device is biocompatible. In another aspect, the device is coated with a material that is biocompatible and/or bioactive. In another aspect, the elements are selected from Ag, Al, Mg, Mn, Cd, Co, Cr, Cu, Fe, Ge, Mo, Nb, Ni, Si, Sb, W, and Zn. In another aspect, the elements are selected from Co, Cr, Fe, Ni, Mn, and Al.

Another embodiment of the present invention includes a method of making a biomedical device comprising forming a device adaptable for biomedical use; and providing that at least a portion of the device comprises a high entropy alloy comprising at least 5 elements. In one aspect, device is a stent, a valve, a plate, a plaque, a screw, a spring, a tube, a filter, an insert, one or more wires, or a joint. In another aspect, the method further comprises the step of selecting the high entropy alloy from at least one of CoₓCrₓFeₓNiₓMnₓ, or AlₓCoₓCrₓFeₓNiₓ, wherein x is 0.05 to 2. In another aspect, the method further comprises the step of forming the high entropy alloy by at least one of cold rolled, sintered, heat-treated, or microhardened. In another aspect, the method further comprises the step of forming the high entropy alloy by at least one of heat-treating, rolling, and/or heat-treating, wherein the first and second heat-treatment are for the same time and same temperature, or for a different time and temperature. In another aspect, the method further comprises the step of selecting the high entropy alloy from Al_{0.1}CoCrFeNi or CoCrFeNiMn. In another aspect, the five (5) elements are about equimolar. In another aspect, the method further comprises the step of forming the device as a composite of more than one high entropy alloy. In another aspect, the materials for the device are selected to be biocompatible. In another aspect, the method further comprises the step of coating the device with a material that is biocompatible and/or bioactive. In another aspect, the elements are selected from Ag, Al, Mg, Mn, Cd, Co, Cr, Cu, Fe, Ge, Mo, Nb, Ni, Si, Sb, W, and Zn. In another aspect, the elements are selected from Co, Cr, Fe, Ni, Mn, and Al.

Another embodiment of the present invention includes a A biomedical apparatus comprising: a device adapted for use in a biomedical application that comprises a high entropy alloy comprising at least 5 elements selected from Co, Cr, Fe, Ni, Mn, and Al.

### BRIEF DESCRIPTION OF THE DRAWINGS

For a more complete understanding of the features and advantages of the present invention, reference is now made to the detailed description of the invention along with the accompanying figures and in which:

FIG. 1 is a graph that shows a Young's modules of the CoCrFeNiMn high entropy alloys (HEA).

FIGS. 2A to 2C show optical microscopy images of CoCrFeNiMn HEA in (3A) as cast, (3B) heat treated at 700° C. for one hour and (3C) heat treated at 900° C. for one hour shows dendritic microstructure.

FIG. 3A is a graph that shows a Young's modules of CoCrFeNiMn.

FIG. 3B shows an optical microscopy image of CoCrFeNiMn HEA Heat treatment-cold rolling-heat treatment approach was used where the alloy was first heat treated at 1000° C. for 4 hours, cold rolled to 50% and then heat treated at 1000° C. for 4 hours.

FIG. 4 is a graph that compares tensile test was conducted on as-cast HEA and 60% rolled and 1000° C.-24 hours heat-treated samples.

FIG. 5A shows optical microscopy of Al_{0.1}CoCrFeNi as cast, and FIG. 5B shows the Al_{0.1}CoCrFeNi after being rolled and heat-treated.

FIG. 6A is a graph that shows the results E of Al_{0.1}CoCrFeNi.

FIG. 6B is a graph that shows the results E of CoCrFeNiMn.

FIG. 7A is a graph that shows the results of micro-hardness distribution along the weld zones.

FIG. 7B is a graph that shows the results for stress-strain curves.

FIG. 8A shows a structure and material for a conventional stent, as compared to the novel stent of the present invention, which is shown in FIG. 8B.

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

As used herein, the phrase “high entropy alloy” refers to an allow of equal or near equal quantities of 5 or more metals, which have at least one of: superior strength-to-weight ratios, a higher degree of fracture resistance, tensile strength, corrosion and/or oxidation resistance than conventional alloys. Generally, a high entropy alloy has concentrations of the 5 or more metals of between 5 and 35 atomic percent, however, it is also possible to have a high entropy allow with 4 or more metals, which definition is also included herein for certain applications. For example, the high entropy alloys can include elements selected from Ag, Al, Mg, Mn, Cd, Co, Cr, Cu, Fe, Ge, Mo, Nb, Ni, Si, Sb, W, and Zn. In certain specific examples of the present invention the 5 or more elements are selected from Co, Cr, Fe, Ni, Mn, and Al.

The present invention concerns alloys with 5 or more principle elements with nearly equal atomic percentage. It has been found that these materials exhibit superior and unique properties such as high strength, wear resistance, high temperature strength, high-fatigue life and corrosion resistance for many applications. One specific application of the alloys taught herein is in cardiovascular diseases and stent implants.

TABLE 1
Commercially available stent materials

| | Yield | Ultimate | | |
|---|---|---|---|---|
| | strength | strength | Young's | % |
| Material | MPa | MPa | modulus | Elongation |
| Stainless steel | 190 | 490 | 193 | 40 |
| 316L | | | | |
| Cobalt | 241-310 | 793-860 | 210-232 | 20-50 |
| chromium | | | | |
| alloys^{a} | | | | |
| Titanium and | 485-1030 | 550-1100 | 110-116 | 10-15 |
| Titanium alloys^{a} | | | | |
| Nitinol (Super elastic | 70-600 | 1070 | 41-75 | — |
| and shape memory) | | | | |

Alarming failure rates in stent implants. It is reported that about 700,000 stent implant procedures are done in the U.S. annually with about a 50% stent failure rate [1]. Failure occurs due to mechanical forces such as bending, torsion, compression, and elongation during the patient's daily activities and heart pulsation. Despite the latest improvements, the failure rates of vascular stents remain high and alternative designs and/or materials are needed.

Biocompatibility of the material has to be considered as the implants can cause adverse effect on the body. As Co-Cr alloys, stainless steel, NiTi alloys, Ti-6Al-4V, Fe-Mn alloys are commonly used as implants materials. However, as pointed out hereinabove, these materials are known to fatigue and cause device failures. The present invention includes the use of novel materials for biomechanical devices. The superior properties of HEAs can minimize the failure rate of stent implants.

Disclosed herein are novel materials and uses for the same that take advantage of the properties of high entropy alloys, e.g., CoCrFeNiMn and Al_{0.1}CoCrFeNi HEAs, are disclosed herein and their significant improvement of over commercially available stent materials is shown.

Two examples of HEAs were chosen, CoCrFeNiMn and Al_{0.1}CoCrFeNi HEA, which were subjected to series of thermo-mechanical processing. The mechanical properties such as yield strength and ultimate strength were measured using custom made mini-tensile machine. Hardness and optical microscopy were done at each condition. Young's modulus was determined using Nano-indentation machine.

Mechanical properties of CoCrFeNiMn HEA.

The as-cast equimolar CoCrFeNiMn HEA was received from the vendor, and the tensile testing was conducted on as-cast condition to understand the initial condition on 700° C. for one hour and 900° C. for one hour heat treated condition to understand its response to heat treatment. The unusual increase in hardness may be due to the formation of nanostructured multiphase embedded in the matrix. FIG. 1 is a graph that shows a Young's modules of the CoCrFeNiMn high entropy alloys (HEA).

TABLE 2
Materials of the present invention.

| | Yield | Ultimate | | Avg. |
|---|---|---|---|---|
| | strength | strength | % | Hardness |
| Condition | MPa | MPa | Elongation | HV0.3 |
| As received (cast | 208 | 447 | 51 | 144.3 |
| condition) | | | | |
| Annealed 700° C.-1 hour | 235 | 505 | 44 | 169.2 |
| Annealed 900° C.-1 hour | 224 | 515 | 51 | 155.7 |

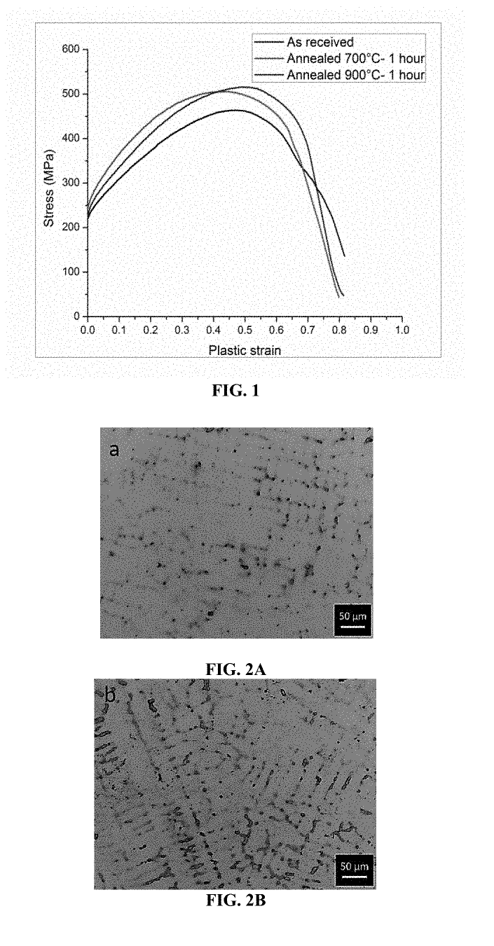

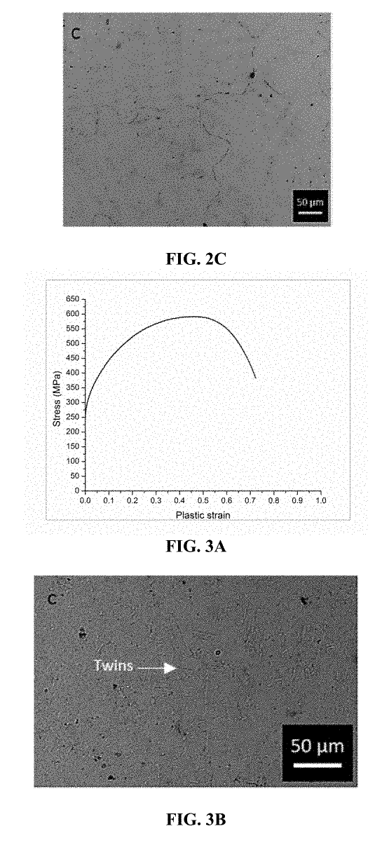

The optical microscopy image of CoCrFeNiMn HEA in (a) as cast, (b) heat treated at 700° C. for one hour and (c) heat treated at 900° C. for one hour shows dendritic microstructure (FIGS. 2A-2C). The size of the dendrites were all most same in all three conditions (Max. 32 μm). Sluggish diffusion was noted. Large number of pores and cast defects were found.

Homogenization by heat treatment and rolling.

Using Fick's law of diffusion and Arrhenius equation concepts the time and temperature required for diffusion was calculated. It was found that this HEA homogenizes after 48 hours of heat treatment at 1200° C. As the chances of oxidation is high when heat treated at 1200° C. and to reduce the pores/cast defects, an alternate approach was decided. It was further found that CoCrFeNiMn HEA heat treatment-cold rolling-heat treatment approach was used where the alloy was first heat treated at 1000° C. for 4 hours, cold rolled to 50% and then heat treated at 1000° C. for 4 hours. FIG. 3A is a graph that shows a Young's modules of CoCrFeNiMn.

FIG. 3B shows an optical microscopy image of CoCrFeNiMn HEA Heat treatment-cold rolling-heat treatment approach was used where the alloy was first heat treated at 1000° C. for 4 hours, cold rolled to 50% and then heat treated at 1000° C. for 4 hours.

Characteristics of homogenized CoCrFeNiMn HEA.

The CoCrFeNiMn HEA after heat treatment-cold rolling-heat treatment turned homogeneous and exhibited twins.

TABLE 3
The CoCrFeNiMn HEA after heat treatment-cold rolling-
heat treatment turned homogeneous and exhibited twins.

| | Yield | Ultimate | | Avg. |
|---|---|---|---|---|
| | strength | strength | % | Hardness |
| Condition | MPa | MPa | Elongation | HV0.3 |
| Heat treated- cold | 273 | 591 | 44 | 157.2 |
| rolled- | | | | |
| heat treated | | | | |

Mechanical properties of Al_{0.1}CoCrFeNi HEA.

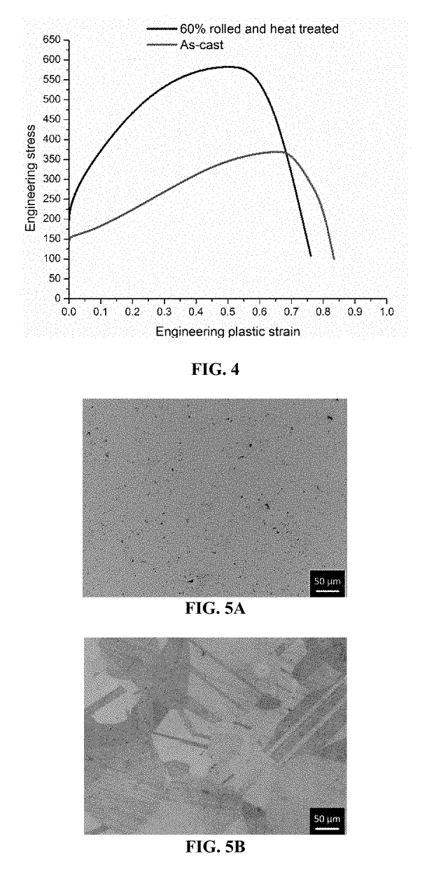

FIG. 4 is a graph that shows the tensile test was conducted on as-cast HEA and 60% rolled and 1000° C.-24 hours heat-treated samples.

TABLE 4
Compares the tensile test was conducted on as-cast HEA and
60% rolled and 1000° C.- 24 hours heat-treated samples.

| | Yield | Ultimate | | Avg. |
|---|---|---|---|---|
| | strength | strength | % | Hardness |
| Condition | MPa | MPa | Elongation | HV0.3 |
| As-cast | 140 | 370 | 65 | 138.2 |
| Rolled and heat | 212 | 570 | 50 | 143.2 |
| treated | | | | |

Optical microscopy of Al_{0.1}CoCrFeNi HEA.

FIG. 5A shows the as cast HEA exhibits coarse grains which causes low yield and ultimate strength. FIG. 5B shows the annealing twins can be found after rolling and heat treatment as this HEA possess low stalking fault energy.

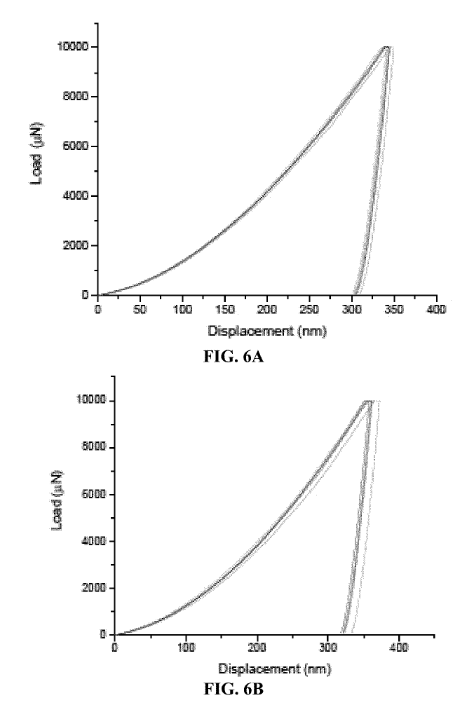

FIGS. 6A and 6B are graphs that show the Determination of Young's modulus.

Young's modulus of CoCrFeNiMn and Al_{0.1}CoCrFeNi HEA were determined using Nano-indentation machine by applying 10000 μN load for 10 seconds.

FIG. 6A is a graph that shows the results for E of Al_{0.1}CoCrFeNi=203 GPa.

FIG. 6B is a graph that shows the results for E of CoCrFeNiMn=189 GPa.

Friction stir processing (FSP) of Al_{0.1}CoCrFeNi HEA. FSP on Al_{0.1}CoCrFeNi HEA [3] at 600 rpm, 1 ipm at 2.5° tool inclination and reported the properties at nugget.

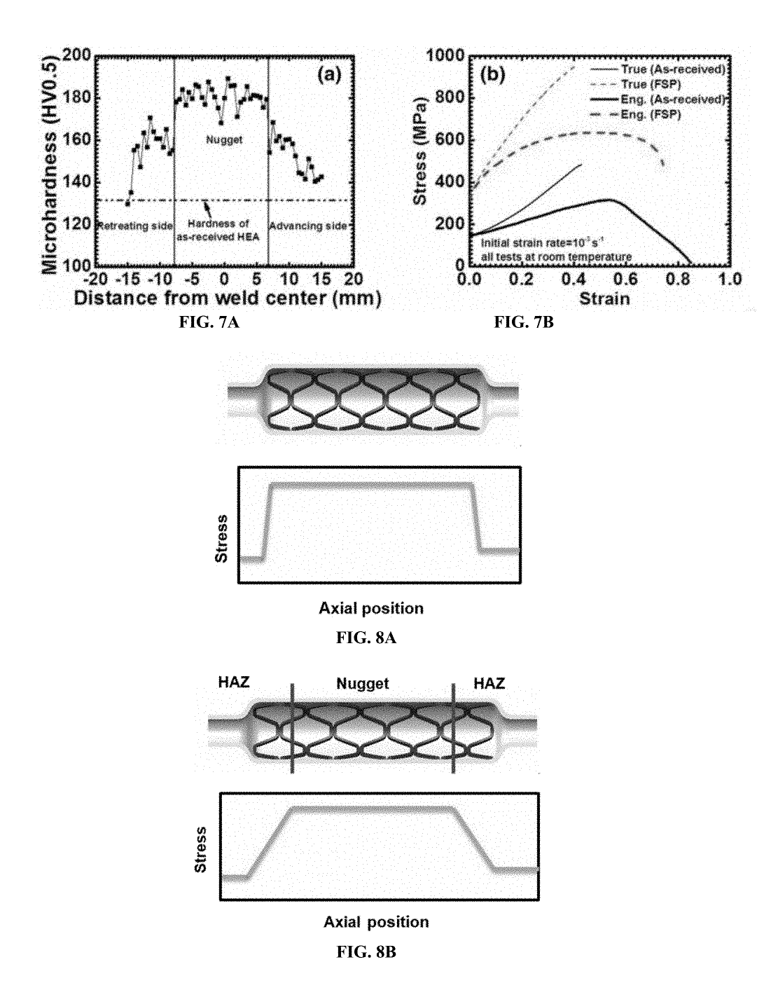

FIG. 7A is a graph that shows the results of micro-hardness distribution along the weld zones.

FIG. 7B is a graph that shows the results for stress-strain curves.

TABLE 5
Friction stir processing of Al_{0.1}CoCrFeNi HEA.

| Yield strength | Ultimate strength | | Avg. Hardness |
|---|---|---|---|
| MPa | MPa | % Elongation | HV0.3 |
| 315 | 600 | 50 | 170 |

One of the advantages of the present invention is shown in the comparison of FIG. 8A and 8B. FIG. 8A shows the structure and materials for a conventional stent. FIG. 8B shows the structure and materials for a biomedical device (a stent is shown) of the present invention.

In conventional stents the strength of the stent is uniform throughout and the stress gradient is high around the edges of the stent. It was found that this leads to undesirable inward motion of tissues at the edges. By using a friction stir processed material to make the stent with high strength/hardness on the nugget and low strength/hardness around the edges minimizes the gradient.

Table 6 is a comparison of the Mechanical properties of the HEA of the present invention versus Stainless steel 316L.

| | | | Ultimate | | |
|---|---|---|---|---|---|
| | | Yield strength | strength | | |
| Material | | (MPa) | (MPa) | E (GPa) | % elongation |
| Stainless steel 316L | | 190 | 490 | 193 | 40 |
| CoCrFeNiMn | As cast | 208 | 447 | 189 | 51 |
| HEA | Heat treated 700° C.-1 | 235 | 490 | 189 | 44 |
| | Hour | | | | |
| | Heat treated 900° C.-1 | 200 | 500 | 189 | 51 |
| | Hour | | | | |
| | 1000° C. 4 hours-cold | 273 | 590 | 189 | 44 |
| | rolled-1000° C. 4 hours | | | | |
| | As cast | 140 | 370 | 203 | 65 |
| Al0.1CoCrFeNi | 60% cold rolled and | 212 | 570 | 203 | 50 |
| | 1000° C. 24 hours heat | | | | |
| | treatment | | | | |
| | Friction stir processed | 315 | 600 | 203 | 50 |

As such, the mechanical properties of CoCrFeNiMn and Al_{0.1}CoCrFeNi are better when compared to stainless steel.

Rolled and heat treated Al_{0.1}CoCrFeNi HEA demonstrates high ultimate stress. The properties can be enhanced to a greater extent by grain refinement techniques like friction stir processing. Thus, friction stir processed stent minimize the stress gradient around the edges.

Fatigue life of CoCrFeNiMn and Al_{0.1}CoCrFeNi HEA can be determined and compared with conventional stent material to prove that the HEAs has better life. The stent implant operation and pulsation of artery due to heart-beat will be simulated using ANSYS. The mechanical properties of HEA found using the experiments will be added to ANSYS library so as to simulate the condition for HEAs. The fatigue life due to systolic and diastolic pressures can also be determined and compared with conventional stent materials. Further, the mechanical properties of each weld zones will be determined and a virtual friction stir processed stent will be simulated using ANSYS.

It is contemplated that any embodiment discussed in this specification can be implemented with respect to any method, kit, reagent, or composition of the invention, and vice versa. Furthermore, compositions of the invention can be used to achieve methods of the invention.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. In embodiments of any of the compositions and methods provided herein, “comprising” may be replaced with “consisting essentially of” or “consisting of”. As used herein, the phrase “consisting essentially of” requires the specified integer(s) or steps as well as those that do not materially affect the character or function of the claimed invention. As used herein, the term “consisting” is used to indicate the presence of the recited integer (e.g., a feature, an element, a characteristic, a property, a method/process step or a limitation) or group of integers (e.g., feature(s), element(s), characteristic(s), propertie(s), method/process steps or limitation(s)) only.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

As used herein, words of approximation such as, without limitation, “about”, “substantial” or “substantially” refers to a condition that when so modified is understood to not necessarily be absolute or perfect but would be considered close enough to those of ordinary skill in the art to warrant designating the condition as being present. The extent to which the description may vary will depend on how great a change can be instituted and still have one of ordinary skilled in the art recognize the modified feature as still having the required characteristics and capabilities of the unmodified feature. In general, but subject to the preceding discussion, a numerical value herein that is modified by a word of approximation such as “about” may vary from the stated value by at least ±1, 2, 3, 4, 5, 6, 7, 10, 12 or 15%.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

### REFERENCES

1. W. Rosamond, K. Flegal, K. Furie, A. Go, K. Greenlund, N. Haase, S. M. Hailpern, M. Ho, V. Howard, B. Kissela, S.

2. Kittner, D. Lloyd-Jones, M. McDermott, J. Meigs, C. Moy, G. Nichol, C. O'Donnell, V. Roger, P. Sorlie, J. Steinberger, T. Thom, M. Wilson, Y. Hong, American Heart Association Statistics Committee, and Stroke Statistics Subcommittee. Heart Disease and Stroke Statistics—2008 Update, volume 117. A Report From the American Heart Association Statistics Committee and Stroke Statistics Subcommittee, 2008.

3. B. Schuh , F. Mendez-Martin , B. Volker , E. P. George , H. Clemens , R. Pippan, A. Hohenwarter ,. Mechanical properties, microstructure and thermal stability of a nanocrystalline CoCrFeMnNi high-entropy alloy after severe plastic deformation. Acta Materialia 96(2015) 258-268.

4. N. Kumar, M. Komarasamy, P. Nelaturu, Z. Tang, P. K. Liaw, and R. S. Mishra,. Friction Stir Processing of a High Entropy Alloy A10.1CoCrFeNi, JOM, Vol. 67, No. 5, 2015.

5. Lucas H. Timmins, B S; Clark A. Meyer, B S; Michael R. Moreno, M S; and James E. Moore, Jr., PhD,. Effects of Stent.

6. Design and Atherosclerotic Plaque Composition on Arterial Wall Biomechanics. J ENDOVASC THER, 2008;15:643-654

What is claimed is:

1. A biomedical apparatus comprising:
a device adapted for use in a biomedical application that comprises a high entropy alloy comprising at least 5 elements.

2. The apparatus of claim 1, wherein the device is a stent, a valve, a plate, a plaque, a screw, a spring, a tube, a filter, an insert, one or more wires, or a joint.

3. The apparatus of claim 1, wherein the high entropy alloy is selected from at least one of CoₓCrₓFeₓNiₓMnₓ, or AlₓCoₓCrₓFeₓNiₓ, wherein x is 0.05 to 2.

4. The apparatus of claim 1, wherein the high entropy alloy is at least one or cold rolled, sintered, heat-treated, or microhardened.

5. The apparatus of claim 1, wherein the high entropy alloy is heat-treated, rolled, and then heat-treated, wherein the first and second heat-treatment are for the same time and same temperature, or for a different time and temperature.

6. The apparatus of claim 1, wherein the high entropy alloys are selected from Al_{0.1}CoCrFeNi or CoCrFeNiMn.

7. The apparatus of claim 1, wherein the five (5) elements are about equimolar.

8. The apparatus of claim 1, wherein the device is a composite of more than one high entropy alloy.

9. The apparatus of claim 1, wherein the device is biocompatible, or is coated with a material that is biocompatible and/or bioactive.

10. The apparatus of claim 1, wherein the elements are selected from Ag, Al, Mg, Mn, Cd, Co, Cr, Cu, Fe, Ge, Mo, Nb, Ni, Si, Sb, W, and Zn.

11. The apparatus of claim 1, wherein the elements are selected from Co, Cr, Fe, Ni, Mn, and Al.

12. A method of making a biomedical device comprising
forming a device adaptable for biomedical use; and
providing that at least a portion of the device comprises a high entropy alloy comprising at least 5 elements.

13. The method of claim 12, wherein the device is a stent, a valve, a plate, a plaque, a screw, a spring, a tube, a filter, an insert, one or more wires, or a joint.

14. The method of claim 12, further comprising the step of selecting the high entropy alloy from at least one of CoₓCrₓFeₓNiₓMnₓ, or AlₓCoₓCrₓFeₓNiₓ, wherein x is 0.05 to 2.

15. The method of claim 12, further comprising the step of forming the high entropy alloy by at least one or cold rolled, sintered, heat-treated, or microhardened.

16. The method of claim 12, further comprising the step of forming the high entropy alloy by at least one of heat-treating, rolling, and/or heat-treating, wherein the first and second heat-treatment are for the same time and same temperature, or for a different time and temperature.

17. The method of claim 12, further comprising the step of selecting the high entropy alloy from Al_{0.1}CoCrFeNi or CoCrFeNiMn.

18. The method of claim 12, wherein the five (5) elements are about equimolar.

19. The method of claim 12, further comprising the step of forming the device as a composite of more than one high entropy alloy.

20. The method of claim 12, wherein the device is biocompatible, or is coated with a material that is biocompatible and/or bioactive.

21. The method of claim 12, wherein the elements are selected from Ag, Al, Mg, Mn, Cd, Co, Cr, Cu, Fe, Ge, Mo, Nb, Ni, Si, Sb, W, and Zn.

22. The method of claim 12, wherein the elements are selected from Co, Cr, Fe, Ni, Mn, and Al.

23. A biomedical apparatus comprising:
a device adapted for use in a biomedical application that comprises a high entropy alloy comprising at least 5 elements selected from Co, Cr, Fe, Ni, Mn, and Al.

24. The apparatus of claim 23, wherein the high entropy alloys are selected from Al_{0.1}CoCrFeNi or CoCrFeNiMn.
